(19) United States
(12) Patent Application Publication
(10) Pub. No.: US 2010/0113906 A1
(43) Pub. Date: May 6, 2010

(54) HYBRID BASKET CATHETERS

(75) Inventors: Eric T. Marple, Loxahatchee, FL (US); Nicholas Green, Juniper, FL (US); James F. Brennan, III, Matthews, NC (US)

Correspondence Address: PATTON BOGGS LLP, 8484 WESTPARK DRIVE, SUITE 900, MCLEAN, VA, 22102, US

(73) Assignee: Prescient Medical, Inc., Doylestown, PA (US)

(21) Appl. No.: 12/609,472

(22) Filed: Oct. 30, 2009

Related U.S. Application Data
(60) Provisional application No. 61/111,927, filed on Nov. 6, 2008.

(51) Int. Cl. A61B 6/00; A61M 25/04

(52) U.S. Cl. 600/342; 604/96.01

(57) ABSTRACT

The invention provides hybrid basket-type intravascular catheter probes designed to optimize blood vessel wall contact or close proximity while traversing tortuous curves. Related diagnostic systems and methods are also provided.

### CROSS-REFERENCE TO RELATED APPLICATIONS

This application claims the benefit of U.S. provisional patent application Ser. No. 61/111,927 filed Nov. 6, 2008, which is incorporated by reference herein in its entirety.

### FIELD OF THE INVENTION

The invention relates to the field of side-viewing intravascular catheter probes and more specifically to basket-style intravascular catheter probes.

### BACKGROUND OF INVENTION

Existing “basket-style” multi-arm tissue contact catheters have been described in prior disclosures such as U.S. Pub. No. 2005/0107706, which is incorporated by reference herein in its entirety. These include various multi-arm tissue contact catheters, including various possible embodiments for deployment and retraction of the basket arms for easier delivery and compatibility with differently sized vessels.

The particular configuration of the scanning core of a basket-style catheter may include single or multiple optical fibers for the transmission and/or collection of light from the side walls of a vessel. Alternatively, the scanning core may be an ultrasonic transducer or transducer array. A third option is the combination of optics and ultrasound, combining the best features from gross morphologic measurements (as with IVUS), fine morphologic measurements (as with OCT and variants) as well as analysis of chemical composition using one of the many available modes of tissue spectroscopy (as with Raman spectroscopy, diffuse reflectance, etc.). Other options include, but are not limited to small temperature transducers (e.g., a thermocouple or RTD thermometer probe) for measuring tissue temperature at the site of contact (thermography). Various basket and umbrella-style tissue contact catheters have been designed and manufactured for intravascular thermography and already exist in the art. Intravascular magnetic resonance imaging (MRI) is another possible detection modality that could be well suited for tissue-contact style catheters.

U.S. Pat. No. 6,522,913 discloses systems and methods for visualizing tissue during diagnostic or therapeutic procedures that utilize a support structure that brings sensors into contact with the lumen wall of a blood vessel, and is incorporated by reference herein in its entirety

U.S. Pat. No. 6,701,181 discloses multi-path optical catheters, and is incorporated by reference herein in its entirety.

U.S. Pat. No. 6,873,868 discloses multi-fiber catheter probe arrangements for tissue analysis or treatment, and is incorporated by reference herein in its entirety.

U.S. Pat. No. 6,949,072 discloses devices for vulnerable plaque detection, and is incorporated by reference herein in its entirety.

U.S. Publication No. 2002/0183622 discloses a fiber-optic apparatus and method for the optical imaging of tissue samples, and is incorporated by reference herein in its entirety.

U.S. Publication No. 2003/0125630 discloses catheter probe arrangements for tissue analysis by radiant energy delivery and radiant energy collection, and is incorporated by reference herein in its entirety.

U.S. Publication No. 2004/00176699 discloses basket-type thermography catheters in which each probe arm is independently moveable, and is incorporated by reference herein in its entirety.

U.S. Publication No. 2004/0204651 discloses infrared endoscopic balloon probes, and is incorporated by reference herein in its entirety.

U.S. Publication No. 2004/0260182 discloses intraluminal spectroscope devices with wall-contacting probes, and is incorporated by reference herein in its entirety.

U.S. Publication No. 2005/0054934 discloses an optical catheter with dual-stage beam redirector, and is incorporated by reference herein in its entirety.

U.S. Publication No. 2005/0075574 discloses devices for vulnerable plaque detection that utilize optical fiber temperature sensors, and is incorporated by reference herein in its entirety.

U.S. Publication No. 2005/0165315 discloses a side-firing fiber-optic array probe, and is incorporated by reference herein in its entirety.

U.S. Publication No. 2006/0139633 discloses the use of high wavenumber Raman spectroscopy for evaluating tissue, and is incorporated by reference herein in its entirety.

The basket-style catheters known in the art are not optimized to continuously maintain contact or close proximity with blood vessel walls while traversing tortuous paths. What is needed is a new type of intravascular basket catheter that continuously maintains excellent contact or proximity with the blood vessel wall while traversing a variety of blood vessel geometries.

### SUMMARY OF INVENTION

One embodiment of the invention provides a multi-arm optical intravascular catheter that includes:

a proximal end;

a distal end;

a central axis;

a proximal catheter segment;

a distal interrogation section extending from the distal end of the proximal catheter segment, wherein the interrogation section includes:
a plurality of flexible full-basket probe arms at least substantially uniformly circumferentially spaced from one another extending from the distal end of the proximal catheter segment that in a radially expanded state radially bow out from the central axis and then, proceeding distally, bow back toward the central axis of the catheter,
a plurality of flexible free-end probe arms extending from the distal end of the proximal catheter segment each having a free distal end and at least substantially uniformly circumferentially spaced from one another,
wherein the flexible full-basket probe arms and the flexible free-end probe arms alternate circumferentially; and

a distal insertion segment connected to the distal ends of the flexible full-basket probe arms, not connected to the distal ends of the flexible free-end probe arms,

wherein at least some of the probe arms, for example, each of the flexible free-end probe arms and/or each of the flexible full-basket probe arms, includes disposed at or near the most radially extendable portion of the probe arm a probe element.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 shows a 10-arm hybrid basket catheter embodiment of the invention that includes five full-basket probe arms and five half-basket probe arms, in which the full-basket probe arms are connected to the distal tip of the catheter which is fixably connected to a central guidewire shaft.

FIG. 2 shows an 8-arm hybrid basket catheter embodiment of the invention that includes four full-basket probe arms and four half-basket probe arms, in which the full-basket probe arms are connected to a floating distal tip segment of the catheter.

FIG. 3 shows Raman spectra of cholesterol and cholesterol esters in the high wavenumber Raman region.

### DETAILED DESCRIPTION

The present invention may identify and solve the problem of maintaining contact or close proximity between the probe arms of a basket intravascular catheter and a blood vessel wall while the catheter traverses tortuous curves in the blood vessel. The invention may have discovered that full-basket intravascular catheters while traversing a curve have a tendency to hug the inner side of the curve, while half-basket catheter while traversing a curve have a tendency to splay to the outer portion of the curve. In solution of this problem, the present invention provides hybrid basket catheters including alternating full-basket type probe arms and half-basket type probe arms.

As used herein the term full-basket, full-basket type probe arms and the like refers to probe arms of a basket catheter having a proximal end and a distal end in which the proximal and distal end are radially disposed at or near the shaft of the catheter, i.e., at or near the central axis of the catheter while the portion between the two ends can bow out to contact or near a lumen wall, such as a blood vessel wall.

As used herein the terms free-end probe arms, half-basket, half-basket type probe arms and the like refer to probe arms that begin with a proximal end at or near the shaft of the catheter, i.e., at or near the central axis of the catheter, and proceeding laterally extend radially outward to contact or near a lumen wall, such as a blood vessel wall but which do not reattach at or near the central axis of the catheter via the distal end of the half-basket type probe arms. However, the free-end probe arms may curve back toward the central axis of the catheter. The free-end probe arms may mimic the profile (curvature) of the full-basket probe arms along their coextensive portions. The free-end probe arms may, for example, extend between 50-90% of the length of the full-basket probe arms, such as about 50%, about 55%, about 60%, about 65%, about 70%, about 80%, or about 85%, or about 90% of the full-basket probe arms.

While the embodiments exemplified in the figures illustrate full-basket arms where the viewing portion (apex of the bowed arms) may be about half-way between the proximal and distal ends of the full-basket type probe arms and the viewing portions of the alternating free probe arm are half-basket probe arms, extending half-way or about half-way as far as the full-basket probe arms so that the viewing portions of the full and half-basket probe arms at least substantially coincide along the longitudinal axis of the basket section, the apices and viewing portions need not be half-way or about half-way longitudinally along the full basket section. For example, a skewed full basket section in which the apices (viewing portions) occur about one-third the longitudinal distance from the proximal end of the basket section is also provided by and within the scope of the invention.

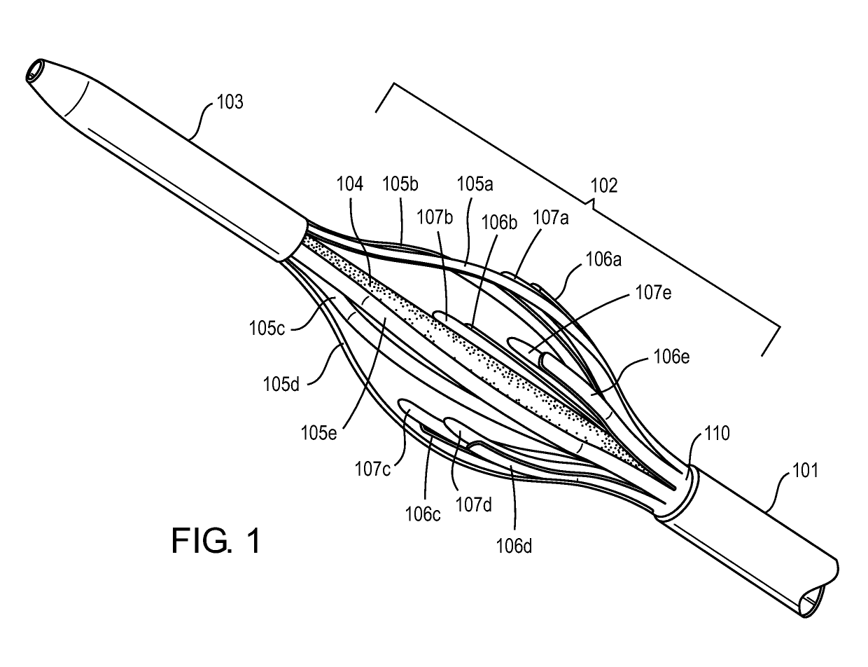

One embodiment of the invention provides a hybrid basket-style catheter in which the full-basket probe arms are connected at the distal end of the proximal segment of the catheter and to a distal end segment (distal tip) that is connected to a longitudinally displaceable internal shaft that runs the length of the catheter so that movement of the internal shaft relative to distal end of the proximal segment of the catheter can be used to control the radial expansion of the full-basket probe arms of the basket section of the hybrid basket catheter. One such embodiment is shown in FIG. 1.

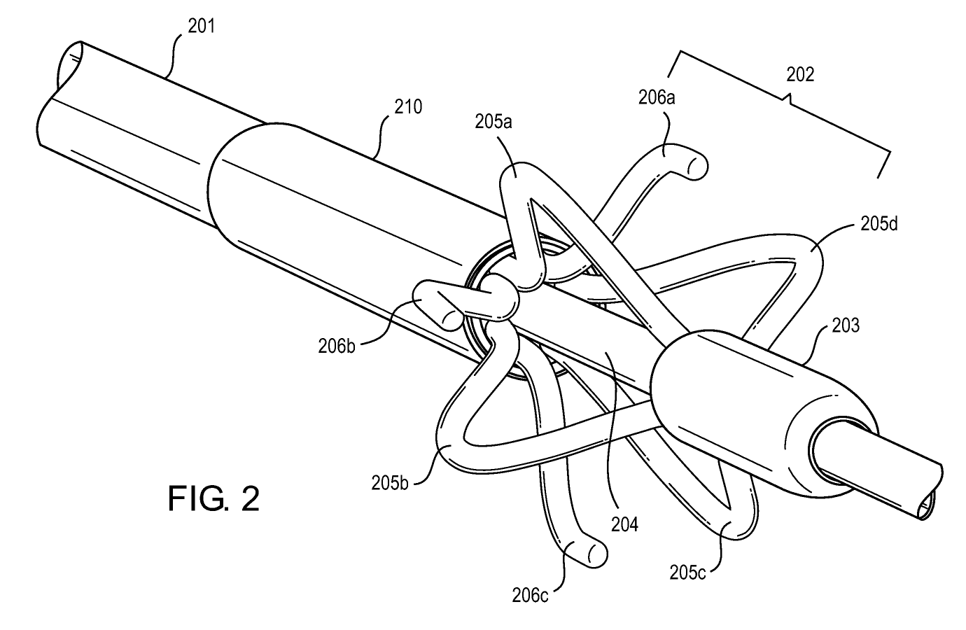

Another embodiment of the invention provides a hybrid basket-style catheter probes in which the full-basket probe arms are configured to automatically radially expand or contract in response to widening or narrowing of a body lumen as the probe traverses the lumen, in the manner of pending co-owned application Ser. No. 11/878,033 (U.S. Pub. No. 2008/0045842), which is incorporated by reference herein in its entirety. One such embodiment is shown in FIG. 2. The basket segment of the probe has a lumen that accommodates a guidewire through its length and includes a proximal end that remains static with respect to the catheter to which it is attached and a distal end that slideably surrounds the guidewire. Positioned between, and attached, to the each of the proximal and distal ends are probe arms that have an outward radial bias so that their tendency is to flex toward a lumen wall. The slideable distal end of the basket segment permits radial expansion and contraction of the probe arms by way of changes in the distance between the proximal and distal ends of the probe as the probe travels within a lumen. The basket section may include a guidewire tube that provides the lumen for the guidewire and about which the distal end segment slideably engages. Alternatively, there may be no separate guidewire tube in the center of the basket section so that a guidewire over which the catheter is passed proceeds through the basket section and the distal end segment directly slideably engages and surrounds the guidewire.

The basket-style probe assemblies of the invention provide for the delivery and/or collection of diagnostic and/or therapeutic energy in small spaces. The probe assemblies can be small and flexible and are well-suited to performing minimally invasive diagnostic examinations of biological tissues in vivo, particularly from within blood vessel lumens

One embodiment of the invention provides a multi-arm optical intravascular catheter that includes:

a proximal end;

a distal end;

a central axis;

a proximal catheter segment;

a distal interrogation section extending from the distal end of the proximal catheter segment, wherein the interrogation section includes:
a plurality of flexible full-basket probe arms at least substantially uniformly circumferentially spaced from one another extending from the distal end of the proximal catheter segment that in a radially expanded state radially bow out from the central axis and then, proceeding distally, bow back toward the central axis of the catheter,
a plurality of free-end probe arms extending from the distal end of the proximal catheter segment each having a free distal end and at least substantially uniformly circumferentially spaced from one another,
   wherein the flexible full-basket probe arms and the free-end probe arms alternate circumferentially; and

a distal insertion segment connected to the distal ends of the flexible full-basket probe arms, not connected to the distal ends of the free-end probe arms,

wherein at least some of the probe arms, for example, each of the free-end probe arms and/or each of the flexible full-basket probe arms, includes disposed at or near the most radially extendable portion of the probe arm a probe element.

In one variation, the curvature of each free-end probe arm in its radially extended state up to the viewing portion thereof may resemble or at least substantially approximate the curvature of the coextensive portion of each full-basket probe arm in its radially extended state.

In another variation, the viewing portions of the full-basket probe arms are longitudinally disposed at or about halfway between the proximal ends and the distal ends of the full-basket probe arms.

In one variation, the distal insertion segment may comprise a guidewire lumen so that the distal insertion segment is slideably engageable with a guidewire. The distal insertion segment may be configured to slideably surround the guidewire. The distal ends of the probe arms may be fixably connected to the distal insertion segment. The proximal catheter segment may also a guidewire lumen (tube) for a guidewire. A control sheath longitudinally extendable and retractable from the distal end of the proximal segment of the catheter to control radial expansion and contraction of the basket section may be provided.

In another variation, the distal insertion segment may be fixably connected to an internal shaft of the catheter that traverses the basket section and proceeds proximally to the proximal end of the catheter, wherein the internal shaft is longitudinally movable with respect to the distal end of the proximal segment of the catheter to control radial expansion and contraction of the basket section.

In one variation, the probe element portions (or viewing portions for optical embodiments) of the full-basket probe arms and the viewing portions of the free-end probe arms at least substantially coincide along the longitudinal axis of the basket section.

The free-end probe arms may each include a probing element in the probing portion, i.e., wall-contacting or wall-nearing portion, thereof. The full-basket probe arms may each include a probing element in the probing portion, i.e., wall-contacting or wall-nearing portion, thereof. Both the free-end and the full-basket probe arms may each include a probing element in the probing portion, i.e., wall-contacting or wall-nearing portion, thereof or only the free-end probe arms or only the full-basket probe arms may include probe elements in the probing portions.

The catheter may be sized and configured for intravascular interrogation of a blood vessel wall, such as a human coronary artery or a human carotid artery.

In another variation, the catheter may include a preformed probe arm reinforcement element consisting of full basket probe arm reinforcement elements and/or free-end probe arm reinforcement elements. The reinforcement element may have a one-piece construction.

The probe elements of the catheter may be of any sort or combinations thereof. In one variation, the probe element may be an IVUS imaging element. In another variation, the probe element may be a thermograph element. The probe elements may also be optical probe elements. In one variation, at least some of the probe arms may include at least one optical fiber entering the probe arm terminating at or near the most radially extendable portion of the probe arm to form a viewing portion of the probe arm capable of transmitting and collecting light. In another variation, each of the probe arms may include at least one optical fiber entering the probe arm terminating at or near the most radially extendable portion of the probe arm, to form a viewing portion of the probe arm capable of transmitting and collecting light. For Raman spectroscopy, in one variation, at least the viewing portions of the probe arms may be enclosed in a polymeric material having at least substantially non-discernable Raman scattering signal in one or more preselected wavenumber regions, used for analysis of a target and being adequately transparent to excitation light delivered via the optical probe element to a Raman-scattered light collected from the illuminated target in the preselected wavenumber range by the optical fiber assembly. The preselected wave number range may be within the high wavenumber region or may not be within the high wavenumber region.

The invention is further described below with reference to the appended figures.

FIG. 1 shows a 10-arm hybrid basket catheter embodiment of the invention that includes 5 full-basket probe arms and 5 half-basket probe arms. The catheter includes a proximal catheter segment 101, and proceeding distally, a “basket section” 102 also referred to as the interrogation section, followed by a distal tip segment 103. At the center of basket section 102 is a central guidewire tube 104 (shown stippled) that fixably connects at its distal end to distal tip 103 and runs proximally to the proximal end of the catheter. Guidewire tube 104 and distal tip 103 provide a continuous guidewire lumen so the catheter may travel over a guidewire (not shown) in an “over-the-wire” configuration. Guidewire tube 104 is laterally displaceable with respect to the distal end of the proximal segment of the catheter to control radial expansion and contraction of the full-basket arms of basket section 102. Basket section 102 includes a flexible, unitary support structure 110 that sets the form for five full-basket probe arms 105a-e and five half-basket probe arms 106/107a-e. Each of the half-basket probe arms includes a side-viewing optical fiber assembly 107a-e, such as a polymer tube/rod-embedded side-viewing optical fiber assembly, attached to a half-basket probe arm support structure 106a-e, which is part of unitary support element 110. The optical fiber assemblies 107a-e each include one or more optical fibers that run from the viewing portion of the probe arms at or near the most radially extendible point to the proximal end of the catheter. In the embodiment shown, the full basket probe arms 105a-e do not include probe elements such as optical fibers but the invention provides embodiments in which the full basket probe arms also may include probe elements.

FIG. 2 shows an 8-arm hybrid basket catheter embodiment of the invention that includes 4 full-basket probe arms and 4 half-basket probe arms, in which the full-basket probe arms connect to a distal tip segment of the catheter which slideably surrounds a central shaft to permit automatic radial adjustment as of the basket section as the catheter traverses lumens of varying diameter. The catheter includes a proximal catheter segment 201, and proceeding distally, a “basket section” 202 also referred to as the interrogation section which includes full-basket probe arms 205a-d and half-basket probe arms 206a-d (206d, not visible, is disposed between 205c and 205d), followed by a distal tip segment 203. At the center of basket section 202 is a central guidewire shaft 204 that extends through an axial lumen in distal tip segment 203 so that the distal tip segment is slideably engaged with shaft 204. This permits the radial extension of the full-basket arms 205a-d to automatically adjust as basket section 202 traverse a lumen of varying diameters. Guidewire shaft 204 includes a guidewire lumen so that the catheter may be run over a guidewire in an “over-the wire” configuration. Retaining sleeve 210 is provided to retain the components of the basket section at its proximal end. Retaining sleeve 210 may optionally be selectively laterally displaceable to act as control sheath or a separate overlying laterally displaceable control sheath may be provided (not shown) to selectively radially compress or release both the half-basket probe arms and the full-basket probe arms of basket section 202. In the embodiment shown, at least one optical fiber runs from the proximal end of the catheter up into each of the probe arms and terminates in a viewing configuration or assembly at or near the most radially extendible portion of the probe arm.

The invention also may provide embodiments similar to that of FIG. 2 wherein there is no guidewire tube 204, so that the basket section including distal tip segment 203 directly surrounds a guidewire over which the catheter is passed.

As referred to herein, the term “probe arm” means one of the flexible elements that is disposed between the proximal end and distal end of the basket section and which contacts or nears a lumen wall, such as an artery wall, by radial extension. One or more of the probe arms may include an operable probe element or sensor, also referred to as a scanning core herein, for delivering and/or receiving diagnostic or therapeutic energy, for example, light, ultrasound or heat. A 4-channel basket catheter profile is shown in the figures. However, catheters of the invention may generally have at least four probe arms and may, for example, have 4, 5, 6, 7, 8, 9 or 10 probe arms where the probe arms alternate between free-end probe arms and full-basket probe arms. By using multiple circumferentially spaced probe arms, a composite radial field-of-view can be built up. The probe arms may be at least substantially uniformly circumferentially spaced.

The particular configuration shown in the accompanying figures is an “over the wire” catheter with a guidewire lumen passing the entire length of the catheter, and out through the “guidewire port” on the hub. For simplicity, the remaining descriptions will discuss the embodiment as an optical spectroscopy catheter, but the invention is not limited to this modality and may, for example, be additionally or alternatively implemented with other diagnostic modalities such as ultrasound (IVUS), MRI, OCT or thermography.

The optical fiber bundles may begin within each distal scanning optic core and extend to and/or beyond the proximal end of the catheter to connectors which interface with a light source and detector. Each optical fiber bundle may contain one or more optical fibers.

U.S. Publication No. 2004/00176699 teaches embodiments of a basket type thermography catheter in which the distal ends of each probe arm are independently slideably engaged with the distal tip segment of the catheter. In contrast, the catheters of the present invention are preferably configured to perform optical spectroscopy, such as Raman spectroscopy, such as high wavenumber Raman spectroscopy. Thus, according to the present invention, one or more of the probe arms may contain at least one optical fiber for side/lateral-viewing from the scanning core regions of the probe arms. Further in contrast to U.S. Publication No. 2004/00176699, according to the present invention, the distal end of each probe arm may be fixably connected (have a fixed connection point) to or integrated with the distal tip segment of the catheter, thereby simplifying construction and operation of the catheter.

For optical probe elements, a lateral field-of-view may be provided by any suitable means, for example, by using a mirror or prism in optical communication with the one or more optical fibers and/or by using angle-cut optical fiber faces. For example, a 45-degree mirror or prism may be used to laterally redirect light with respect to a distal scanning core of a probe.

The outward radial shape or “bias” of the probe arms for tissue contact may, for example, be obtained by utilizing probe arms with a pre-set curvature. For example, the probe arms may be formed from plastic/polymer tubing or segments having a curvature that provides the outward radial shape for tissue contact. Another approach is to provide this support via a structural support element. The support/reinforcement member may be a unitary structure, i.e., a one-piece structure, as shown in FIG. 1. The support structure may, for example, be made from a stainless steel, a spring steel, superelastic Nitinol alloy or a polymeric material such as PEEK, Polyimide, Polyamide, an amorphous fluoropolymer such as TEFLON AF (Dupont), polychlorotrifluoroethylene (PCTFE) or other engineering materials for medical device construction. If polytetrafluoroethylene (PTFE) is used, it is preferably not used alone but with a separate support material, for example, an underlying nitinol frame, due to the softness of PTFE. The basket reinforcement element tube may, for example be fabricated in a collapsed form (laser cut thin-walled tubing) and then compressed (with respect to its lateral axis) within a mold base and heat treated to set the preferred unconstrained shape. Injection molding or thermoforming of plastic/polymer materials may also be used to fabricate the basket reinforcement element.

The invention also may provide a method for diagnostically interrogating and/or treating a body lumen wall, such as blood vessel lumen wall, that includes the steps: of inserting a catheter according to the invention into a body lumen, such as a blood vessel lumen; and delivering diagnostic and/or therapeutic energy via at least one probe element on at least one probe arm of the catheter to the lumen wall. The energy may for example, be light energy. Energy received via the probe elements or measured by the probe elements may be analyzed to evaluate and diagnose a subject tissue. The invention is not limited by the method used to interrogate and diagnosis the condition of a blood vessel wall. Optical and/or non-optical methods may be used. Multiple methods may also be used. Suitable optical methods include, but are not limited to, low-resolution and high resolution Raman spectroscopy, fluorescence spectroscopy, such as time-resolved laser-induced fluorescence spectroscopy, reflectance spectroscopy, absorption spectroscopy and laser speckle spectroscopy. Photoacoustic stimulation in conjunction with acoustical detection by any means may also be used. Another embodiment of the invention is a method for diagnosing and/or locating one or more atherosclerotic lesions, such as vulnerable plaque lesions, in a blood vessel, such as a coronary artery of a subject, using a catheter as described herein to evaluate the properties of a vessel wall, such an artery, at one or more locations along the vessel. In any of the embodiments, the catheter including its basket section and probe arms thereof may be sized for interrogation of human coronary arteries.

Differentially diagnosing, identifying and/or determining the location of an atherosclerotic plaque, such as a vulnerable plaque, in a blood vessel of a patient may be performed by any method or combination of methods. For example, catheter-based systems and methods for diagnosing and locating vulnerable plaques may be used, such as those employing optical coherent tomography (“OCT”) imaging, temperature sensing for temperature differentials characteristic of vulnerable plaque versus healthy vasculature, labeling/marking vulnerable plaques with a marker substance that preferentially labels such plaques, infrared elastic scattering spectroscopy, and infrared Raman spectroscopy (IR inelastic scattering spectroscopy). U.S. Publication No. 2004/0267110 discloses a suitable OCT system and is hereby incorporated by reference herein in its entirety. Raman spectroscopy-based methods and systems are disclosed, for example, in: U.S. Pat. Nos. 5,293,872; 6,208,887; and 6,690,966; and in U.S. Publication No. 2004/0073120, each of which is hereby incorporated by reference herein in its entirety. Infrared elastic scattering based methods and systems for detecting vulnerable plaques are disclosed, for example, in U.S. Pat. No. 6,816,743 and U.S. Publication No. 2004/0111016, each of which is hereby incorporated by reference herein in its entirety. Time-resolved laser-induced fluorescence methods for characterizing atherosclerotic lesions are disclosed in U.S. Pat. No. 6,272,376, which is incorporated by reference herein in its entirety. Temperature sensing based methods and systems for detecting vulnerable plaques are disclosed, for example, in: U.S. Pat. Nos. 6,450,971; 6,514,214; 6,575,623; 6,673,066; and 6,694,181; and in U.S. Publication No. 2002/0071474, each of which is hereby incorporated by reference herein in their entirety. A method and system for detecting and localizing vulnerable plaques based on the detection of biomarkers is disclosed in U.S. Pat. No. 6,860,851, which is hereby incorporated by reference herein in its entirety.

Raman spectroscopy has proven capable of determining the chemical composition of tissues and diagnosing human atherosclerotic plaques. Typical methods of collecting Raman scattered light from the surfaces of artery do not register information about how far the scattering element is from the collection optics. Two wavenumber regions that yield useful information for evaluating the condition of blood vessels are the so-called Raman fingerprint region i.e., approximately 200 to 2,000 cm⁻¹, and the so-called high wavenumber region, i.e., approximately 2,600 to 3,200 cm⁻¹. The collection of Raman spectra in the fingerprint (FP) region, through optical fibers is complicated by Raman “background” signal from the fibers themselves. In order to collect uncorrupted FP spectra, complicated optics and filters on the tips of catheters and often these designs require the use of multiple fibers. Because the Raman scattered signal is weak, large multimode fibers are utilized in the multi-fiber catheter designs, which creates an unwieldy catheter that is less than optimal for exploring delicate arteries, such as the human coronary arteries. However, common optical fiber materials generate very little Raman background signal in the high wavenumber region, permitting a simplified, single optical fiber probe element implementation of intravascular Raman spectroscopy.

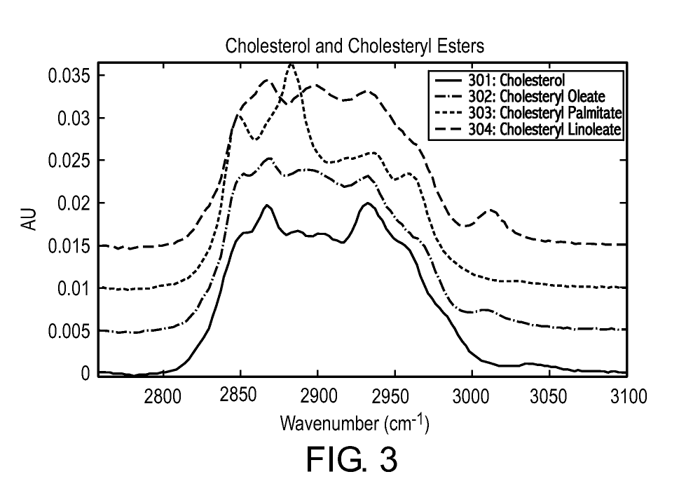

Because cholesterol and its esters have distinctive Raman scattering profiles within the Raman high wavenumber region, the use of the Raman high wavenumber region for analysis is particularly useful for locating and characterizing lipid-rich deposits or lesions that may occur in blood vessels, such as vulnerable plaques in arteries, for example, the coronary arteries. FIG. 3 shows Raman spectra of cholesterol and cholesteryl esters in the high wavenumber region. Specifically, curve 301 is a Raman spectrum for cholesterol, curve 302 is a Raman spectrum for cholesteryl oleate, curve 303 is a Raman spectrum for cholesteryl palmitate and curve 304 is a Raman spectrum for cholesteryl linolenate.

Another embodiment of the invention may provide a method for evaluating the wall of a blood vessel such an artery, such as a coronary artery, such as a human coronary artery, that includes the steps of:

providing any of the intravascular basket catheter embodiments of the invention having a proximal end and a distal insertion end including a hybrid basket section that includes full-basket probe arms and free-end probe arm;

disposing the hybrid basket section of the catheter in a blood vessel; and

taking optical readings of the vessel wall at one or more locations in the blood vessel using via the probe arm of the hybrid basket section.

In another variation, the method may include transmitting light, for example, laser light, from a light source to target regions of a lumen wall, such as a blood vessel wall, via the scanning core of the probe arms of the catheter and collecting and analyzing inelastically scattered (Raman scattered) light resulting from the illumination of the target regions using a Raman spectrometer. The Raman spectrometer may be configured to measure Raman scattered light in the high wavenumber region and/or the fingerprint region and the data for either or both of the regions may be analyzed to determine the chemical composition of the target regions and/or diagnose the target regions/tissue.

In another variation, the method may include transmitting light, for example, laser light, for fluorescence stimulation of the target regions of a lumen wall, such as a blood vessel wall, via the scanning core of the probe arms of the catheter and collecting and analyzing fluorescent emissions resulting from the illumination of the target regions using a fluorescence spectrometer. In a sub-variation, time-resolved laser-induce fluorescence may be performed using a catheter embodiment of the invention.

It should be understood for the above methods that the probe arms may be radially extended to contact or closely near the vessel walls in order to take readings from the probe arm viewing portions. The step of taking readings may include taking the recited readings at more than one longitudinal location in a blood vessel, for example, while the catheter is pulled back by operation of a catheter pullback mechanism.

The invention also may provide an integrated system for evaluating the status of a lumen wall, such as a blood vessel wall, for example, for diagnosing and/or locating vulnerable plaque lesions, that includes a basket-style catheter according to the invention having probe elements (scanning cores) for interrogating the lumen wall that are in communication with an analyzer for analyzing the signal and/or information received via the probe elements. The analyzer may include a computer.

A related embodiment may provide an integrated system for optically evaluating the status of a lumen wall, such as a blood vessel wall, for example, for diagnosing and/or locating atherosclerotic lesions, such as vulnerable plaque lesions in an artery, that includes a basket-style catheter according to the invention, having optical probe elements for interrogating the blood vessel in communication with a light source, such as a laser for illuminating a target region of a blood vessel via the catheter and a light analyzer, such as a spectrometer, for analyzing the properties of light received from the target region via the catheter.

A related embodiment of the invention may provide a diagnostic catheter system for the evaluation of blood vessel walls that may include an intravascular diagnostic catheter as described herein, a light source, such as a laser for stimulating Raman scattered light emissions from a target region via the wall-contacting portion (scanning core) of the probe arms of the catheter, and a Raman spectrometer for analyzing Raman scattered light collected from a target via the wall-contacting portion of the probe arms of the catheter. The system may be configured to collect and analyze Raman spectral data within the region of approximately 2,600 to 3,200 cm⁻¹, i.e., the so-called high wavenumber region, and/or the within the region of approximately 200 to 2,000 cm⁻¹, i.e., the so-called fingerprint region. The optical probe arms may, for example, each have a single optical fiber and the system may be configured to perform high wavenumber Raman spectroscopy from each probe arm via the single optical fiber.

One or more computers, or computer processors generally working in conjunction with computer accessible memory, may be part of any of the systems for controlling the components of the system and/or for analyzing information obtained by the system.

Co-owned U.S. patent application Ser. No. 11/876,899 (U.S. Pub. No. 2008/0129993) which is hereby incorporated by reference in its entirety, teaches windowless optical probe assemblies for use with Raman spectroscopy, such as high wavenumber Raman spectroscopy, which may be implemented with the present invention. In accordance with this application, the probe arms may include one or more optical fibers housed in material(s) having a very low Raman scattering cross-section in the wavenumber region used for analysis of a target and being adequately transparent to excitation light delivered via the fiber optics to the target and to Raman-scattered light (inelastically scattered light) collected from the irradiated target in the desired wavenumber range. In this manner, a separate window or aperture is not needed in the viewing portion (scanning core region) of the probe arm to permit illumination and collection of light having the desired ranges of wavelengths, thereby simplifying manufacture and improving performance of the catheter.

Thus, in any of the embodiment of the present invention, at least one of the probe arms may include: an optical fiber assembly having a viewing portion (scanning core portion) for transmitting and receiving light, wherein at least the viewing portion of the optical fiber assembly is enclosed in a material, such as a polymeric material, having an at least substantially non-discernable Raman scattering signal (a level not interfering with analysis) in one or more preselected wavenumber regions used for analysis of a target and being adequately transparent to excitation light delivered via the optical fiber assembly to the target and to Raman-scattered light collected from the irradiated target in the preselected wavenumber range by the optical fiber assembly. The optical fiber assemblies of the probes and catheter probes may include one or more optical fibers. In one variation, the main bodies of the probe arms (excluding the optical fiber assemblies) may be entirely composed of or enclosed in the polymeric material. The preselected wavenumber region may, for example, be in the range of approximately 2,600 to 3,200 cm-1, i.e., within the high wavenumber region. For the high wavenumber region, the enclosure material may, for example, include or consist of polymer material that at least substantially does not include carbon-hydrogen bonds, such as an amorphous fluoropolymer (such as TEFLON AF (Dupont)), polychlorotrifluoroethylene (PCTFE), polytetrafluoroethylene (PTFE), fluorinated ethylene-propylene (FEP) and perfluoroalkoxy polymer resin (PFA). In these cases, the excitation wavelength used to obtain the high wavenumber spectra may, for example, be at or around 740 nm, or at a suitable near infrared wavelength generally. In one embodiment, light at or around 671 nm is used. Generally, a wavelength of light that is absorbed only to a low degree by blood and water may be used. Any suitable light source may be used. For example, the light source may be a laser. One suitable type of laser light source may be a diode-pumped solid state lasers (DPSS), such as those known in the art. Another suitable type of laser light source may be a wavelength stabilized multi-mode laser diode, such as a Volume Bragg Grating Stabilized multi-mode laser diode (available, e.g., from PD-LD, Inc., Pennington, N.J.)

Each of the patents and other publications cited in this disclosure is incorporated by reference in its entirety.

Although the foregoing description is directed to the preferred embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1. An optical intravascular catheter, comprising:
a proximal end;
a distal end;
a central axis;
a proximal catheter segment;
a distal interrogation section extending from the distal end of the proximal catheter segment, wherein the interrogation section comprises:
a plurality of flexible full-basket probe arms at least substantially uniformly circumferentially spaced from one another extending from the distal end of the proximal catheter segment that in a radially expanded state radially bow out from the central axis and then, proceeding distally, bow back toward the central axis of the catheter,
a plurality of flexible free-end probe arms extending from the distal end of the proximal catheter segment each having a free distal end and at least substantially uniformly circumferentially spaced from one another,
wherein the flexible full-basket probe arms and the flexible free-end probe arms alternate circumferentially; and
a distal insertion segment connected to the distal ends of the full-basket probe arms, not connected to the distal ends of the flexible free-end probe arms,
wherein each of the flexible full-basket probe arms and/or each of the flexible free-end probe arms comprises at least one optical fiber entering the probe arm and terminating at or near the most radially extendable portion of the probe arm in a configuration or assembly to form a viewing portion of the probe arm capable of transmitting and collecting light.

2. The catheter of claim 1, wherein the curvature of each flexible free-end probe arm in its radially extended state up to the viewing portion thereof resembles the curvature of the coextensive portion of each flexible full-basket probe arm in its radially extended state.

3. The catheter of claim 1, wherein the viewing portions of the flexible full-basket probe arms are longitudinally disposed at or about halfway between the proximal ends and the distal ends of the flexible full-basket probe arms.

4. The catheter of claim 1, wherein the distal insertion segment comprises a guidewire lumen so that the distal insertion segment is slideably engageable with a guidewire.

5. The catheter of claim 2, wherein the distal ends of the flexible full-basket probe arms are fixably connected to the distal insertion segment.

6. The catheter of claim 3, wherein the proximal catheter segment comprises a guidewire lumen for a guidewire.

7. The catheter of claim 1, further comprising a control sheath longitudinally extendable and retractable from the distal end of the proximal segment of the catheter to control radial expansion and contraction of the basket section.

8. The catheter of claim 1, wherein the distal insertion segment is fixably connected to an internal shaft of the catheter that traverses the basket section and proceeds proximally to the proximal end of the catheter, wherein the internal shaft is longitudinally movable with respect to the distal end of the proximal segment of the catheter to control radial expansion and contraction of the basket section.

9. The catheter of claim 1, wherein the viewing portions of the flexible full-basket probe arms and the viewing portions of the flexible free-end probe arms at least substantially coincide along the longitudinal axis of the basket section.

10. The catheter of claim 2, wherein the viewing portions of the flexible full-basket probe arms and the viewing portions of the flexible free-end probe arms at least substantially coincide along the longitudinal axis of the basket section.

11. The catheter of claim 8, wherein the viewing portions of the flexible full-basket probe arms and the viewing portions of the flexible free-end probe arms at least substantially coincide along the longitudinal axis of the basket section.

12. The catheter of claim 2, wherein the distal insertion segment is configured to slideably surround the guidewire.

13. The catheter of claim 1, wherein the catheter is sized and configured for intravascular interrogation of a blood vessel wall.

14. The catheter of claim 13, wherein the blood vessel wall is a human coronary artery wall.

15. The catheter of claim 1, further comprising a preformed probe arm reinforcement element consisting of full basket probe arm reinforcement elements and free-end probe arm reinforcement elements.

16. The catheter of claim 1, wherein at least the viewing portions of the probe arms are enclosed in a polymeric material having at least substantially non-discernable Raman scattering signal in one or more preselected wavenumber regions used for analysis of a target and being adequately transparent to excitation light delivered via the optical probe element to Raman-scattered light collected from the illuminated target in the preselected wavenumber range by the optical fiber assembly.

17. The catheter of claim 16, wherein the preselected wave number range is or is within the high wavenumber region.

18. An optical intravascular catheter system, comprising:
an intravascular catheter according to any one of the preceding claims;
at least one light source in optical communication with the viewing portion of each probe arms wherein said light source is suitable for generating Raman spectra; and
a Raman spectrometer in optical communication with the viewing portion of each probe arm.

19. The system of claim 18, further comprising at least one computer processor.

20. An intravascular catheter, comprising:
a proximal end;
a distal end;
a central axis;
a proximal catheter segment;
a distal interrogation section extending from the distal end of the proximal catheter segment, wherein the interrogation section includes:
a plurality of flexible full-basket probe arms at least substantially uniformly circumferentially spaced from one another extending from the distal end of the proximal catheter segment that in a radially expanded state radially bow out from the central axis and then, proceeding distally, bow back toward the central axis of the catheter,
a plurality of flexible free-end probe arms extending from the distal end of the proximal catheter segment each having a free distal end and at least substantially uniformly circumferentially spaced from one another,
wherein the flexible full-basket probe arms and the flexible free-end probe arms alternate circumferentially; and
a distal insertion segment connected to the distal ends of the full-basket probe arms, not connected to the distal ends of the free-end probe arms.

21. The catheter of claim 20, wherein at least one of the probe arms includes a probe element disposed at or near the most radially extendable portion of the probe arm.

22. The catheter of claim 21, wherein each of the flexible full-basket probe arms and/or each of the flexible free-end probe arms comprises at least one probe element.
